(19) United States
(12) Patent Application Publication
(10) Pub. No.: US 2005/0255556 A1
(43) Pub. Date: Nov. 17, 2005

(54) Refolding of ion channel proteins

(76) Inventors: Dietrich Loebel, Filderstadt (DE); Tilmann Roos, Kusterdingen (DE); Klaus Maier, Stuttgart (DE); Hans Kiefer, Tuebingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/102,487

(22) Filed: Apr. 8, 2005

Related U.S. Application Data
(63) Continuation of application No. PCT/EP03/10035, filed on Sep. 10, 2003.

(30) Foreign Application Priority Data
Oct. 11, 2002 (DE) 102 48 123.7

(51) Int. Cl. C07K 14/705; C12Q 1/68; C07H 21/04; C12P 21/06; C12N 15/09

(52) U.S. Cl. 435/69.1; 435/320.1; 435/325; 530/350; 536/23.5

(57) ABSTRACT

A method of ion channels folded into their active structure, or functional subunits thereof, is described. For this purpose, initially expressed subunits, which are solubilized and denatured in a first detergent, of an ion channel are provided, and subsequently the first detergent is replaced by a second detergent which induces folding of the subunits of an ion channel into their native structure. The subunits of the ion channel are then assembled into its active structure.

### CROSS REFERENCES TO RELATED APPLICATIONS

This application is a continuation of co-pending international application PCT/EP03/10035 filed on Sep. 10, 2003 and designating the U.S., which was not published under PCT Article 21(2) in English, and claims priority of German patent application DE 102 48 123.7 filed on Oct. 11, 2002, which is incorporated herein by reference.

### BACKGROUND OF THE INVENTION

1. Field of the Invention

The present invention relates to a method for preparing ion channels which are folded into their active structure, or functional subunits thereof, to the use of a detergent for preparing ion channels or functional subunits thereof, and to a method for determining the activity of ion channels.

2. Related Prior Art

Methods for refolding denatured proteins into their native structure are generally known in the art.

Biological cells are surrounded by a lipid bilayer membrane which, owing to its lipophilic property, is impermeable to water and ions. In order to enable the cell to exchange ions with its surroundings, so-called channel proteins, which are also referred to as ion channels, are present in the cell membrane. These ion channels form an annular channel pore through the cell membrane, which can be temporarily opened.

Factors relevant to the selectivity of the ions which pass through the respective ion channels are frequently charged amino acid residues of the inner wall of the pores, and the atomic radius, the hydration sheath and the electric charge of the ion.

Ion channels are ordinarily composed of a plurality of subunits. These subunits may have the same structure (homomers), as for example in the case of the glycine channel, or the channel consists of different subunits (heteromers), like for example the acetylcholine receptor.

Ion channels can be classified for example descriptively, i.e. according to the ionic species for which they are permeable. Another important classification is based on the mechanism of control of the channels, i.e. which signals lead to opening or closing of the pores. A distinction is made in this connection between voltage-gated and ligand-gated channels. The voltage-gated ion channels, e.g. voltage-gated sodium channels, potassium channels and calcium channels, are of crucial physiological importance. They are characterized by a change in the electric field of the cell membrane leading to them opening. The ligand-gated ion channels include for example nicotinic acetylcholine receptor channels, glutamate receptor channels, serotonin receptor channels, glycine receptor channels or GABA receptor channels. These ion channels are important in particular for rapid synaptic transmission. Ligand-gated ion channels are characterized by the binding of their specific ligands or of analogs thereof leading to opening of the channels.

It is known that dysfunctions of ion channels are involved in a large number of diseases. Thus, mutations have recently been found in two previously unknown brain-specific potassium channels. The reduced flow of potassium due to the mutations causes a hyperexcitability of certain neurons, possibly leading to clinically epileptic seizures. In addition, a genetic defect in a calcium channel has been found to induce familial hemiplegic migraine. Changes in ion channels or in individual subunits play an important part also in the development of congenital generalized or local myasthenia, certain types of ventricular fibrillation or deafness.

With this background, the investigation of ion channel proteins has considerable importance in particular also in connection with the finding of novel pharmacological active ingredients. The basis for in vitro investigations on ion channels is formed by the provision of isolated, purified and, in particular, functionally active ion channel proteins in quantities which are as large as possible. Suitable methods for preparing native ion channel proteins are indispensable therefor.

Various methods for preparing proteins of a wide variety of types are known in the art. Besides the purification of proteins from biological material in which they naturely occur, also known are genetic manipulation methods in which proteins are expressed in bacterial, mammalian, insect, plant or yeast cells and subsequently purified.

Thus, Graham et al., Purification and Characterization of the Glycine Receptor of Pig Spinal Cord, Biochemistry (1985), 24, 990-994, describe the purification of a glycine receptor channel from membranes of the spinal cord of the domestic pig. However, the method described therein is very complicated, inter alia requiring very large quantities of tissue. The method moreover cannot be applied directly to other ion channel proteins. The yield of naturely folded receptor is also very low. The described method is therefore unsuitable for large-scale use.

Cascio et al., Functional Expression and Purification of a Homomeric Human α1 Glycine Receptor in Baculovirus-infected Insect Cells, The Journal of Biological Chemistry (1993), 268, 22135-22142, describe a method based on a eukaryotic expression system. A human α1 glycine receptor channel is expressed in baculovirus-infected Sf9 insect cells therein. The expressed channel subsequently becomes incorporated into the cell membrane of the insect cells and must then undergo elaborate isolation from the membrane. The method described therein additionally has the disadvantage that the yield of naturely folded channel protein is very low. The reason for this is in particular that high concentrations of channels in the membrane lead to the deaths of the insect cells and thus to expression not occurring. The method described therein is therefore also unsuitable for large-scale use.

Taleb and Betz, Expression of the human glycine receptor α1 subunit in Xenopus oocytes: apparent affinities of agonists increase at high receptor density, The EMBO Journal (1994), 13, 1318-1324, likewise describe a method based on a eukaryotic expression system and allowing a subunit of the human glycine receptor channel to be prepared. For this purpose, cDNA constructs which code for said subunit are injected into Xenopus oocytes. The channel protein subunit is then expressed in the oocytes and incorporated into the oocyte cell membrane. Said document does not describe the further necessary purification of the protein from the membrane. This known method is also very complicated and results in a very low yield of naturely folded protein, so that it is equally unsuitable for large-scale applications.

WO 01/14407 discloses a method for preparing G-protein-coupled receptors (GPCR), in which the GPCR protein is expressed in bacterial cells and is initially in the form of so-called insoluble inclusion bodies. The GPCR are then folded into their nature shape by a detergent exchange. The GPCR are, however, not biochemically comparable to ion channel proteins.

Thus, GPCRs differ considerably in their three-dimensional structure from the channel-forming proteins. Most GPCRs have a conserved secondary structure consisting of seven transmembrane helices with an extracellular N-terminal loop region. GPCRs serve to relay a signal within the signal transduction cascade, but not to transfer an ion through. The structure, differing from the GPCRs, of the ion channel proteins which—as mentioned—ordinarily have a plurality of subunits also results in an entirely different folding behavior of the amino acid chains, i.e. assumption of the nature structure takes place in a different way for ion channel proteins than for GPCRs. Not the least important reason for this is the frequently occurring defined arrangement of charged amino acid residues in the interior of the pore of an assembled channel.

### SUMMARY OF THE INVENTION

Against this background, it is an object of the present invention to provide a method for preparing ion channels which are folded into their active structure, or functional subunits thereof, with which the disadvantages of the prior art are avoided.

This object is achieved according to the invention by such a method comprising the following steps:
(a) provision of expressed subunits of an ion channel which are solubilized and denatured in a first detergent,
(b) replacement of the first detergent by a second detergent which induces folding of the subunits into their native structure, and
(c) assembly of subunits of the ion channel into its active structure.

The above-mentioned object is completely achieved in this way.

This is because the inventors have realized that starting material expressed bacterially or in a cell-free system can also be employed for preparing naturely folded ion channel protein. This has the particular advantage that, compared with the eukaryotic expression systems employed for ion channels to date in the art, very large quantities of protein material are available thereby, and consequently also large quantities of naturely folded ion channel can be obtained. The starting material is usually deposited as inclusion bodies in the cytosol of the bacterial cell, for example E. coli, and is thus non-toxic for the latter. The inclusion bodies can moreover be separated from bacterial protein by simple methods known to the skilled person, e.g. by centrifugation.

In this connection, “expressed” means both a bacterial expression and an in vitro expression in a cell-free system. Bacterial expression systems are sufficiently well-known and tested in the art. For expression in the cell-free system it is possible to employ for example the “Rapid Translation System (RTS)” from Roche Diagnostics GmbH, Mannheim, Germany.

“Functional subunits” means herein that subunits which have the function of an ion channel but without forming a “complete” ion channel are also included.

There has to date been no description or attempt in the art to express for example human ion channel proteins in bacteria or in a cell-free system, because the solubility problems often associated therewith, due to precipitation of the protein as inclusion bodies, have not been solved to date. In particular, no method with which the bacterially expressed and functionally inactive ion channel proteins in the form of inclusion bodies can be folded into their native structure has yet been available to skilled persons.

It is now possible according to the invention for the first time to fold this functionally inactive ion channel protein into its native structure. As the inventors have realized, it is crucial therefor that the inactive protein is denatured and dissolved in a first detergent, and subsequently this detergent is replaced by a further detergent which induces folding of the protein. Accordingly, replacement of a solubilizing by a folding-inducing detergent is crucial for this method. The subunits are assembled so that the ion channel acquires its active structure.

Depending on the intended use of the refolded native channel protein, the method according to the invention may be followed by a step to remove the second detergent, e.g. a dialysis. It may additionally be necessary to form disulfide bridges in the channel protein, e.g. via glutathione.

It is moreover preferred in a further embodiment of the method according to the invention for the ion channel to be a voltage-gated ion channel.

It is preferred in another embodiment for the ion channel to be a ligand-gated ion channel. This is because the inventors have further realized that it is possible with the novel method to prepare such ion channels in the native structure.

It is thus possible with the method according to the invention to prepare channel proteins of particular pharmacological interest, such as, for example, the acetylcholine receptor channel, which appears to play an important part in connection with a particular amyasthenia.

It is further preferred for the subunits to be homomers.

Ion channels composed of homomers can easily be prepared in this way, since the subunits can easily be provided and assembled with the method according to the invention.

It is preferred in a further embodiment for the subunits to be heteromers.

It is likewise possible with the method according to the invention to prepare ion channels composed of different subunits.

In a further development of the method according to the invention, the provision takes place via expression of the subunits as fusion proteins, preferably as a glutathione S-transferase (GST) fusion protein.

The fusion with GST results in the fusion protein, which would on bacterial expression be incorporated “unfused” into the membranes of the bacterial cells, accumulating in the cytosol of the cells.

The fusion with GST makes it particularly easy to handle the protein to be prepared. Thus, the segments fused to the N or C terminus can be used to separate the protein from bacterial protein by coupling to an appropriate column material. It is thus also possible easily to change the buffer or purify the protein. On the other hand, a larger yield is achieved through the GST segment, because—as mentioned—the bacterially expressed ion channel is not integrated into the membrane of the bacterium but, on the contrary, is deposited in the cytosol as inclusion bodies. The fused segments can optionally be cleaved off the folded protein again.

The fusion protein may moreover have a tag with which the fusion protein can be isolated. This may be for example a histidine tag with a certain number of histidines. However, any other tag permitting isolation of the protein tagged therewith can also be employed for this purpose.

In addition, with the method according to the invention it is preferred for step (a) to be followed by a step (a1) in which the subunit binds to a chromatography column material, and for step (b) to be followed by a step (b1) in which the bound subunit is eluted from the chromatography column material.

This has the particular advantage that said detergent exchange takes place thereby particularly simply and in a single reaction mixture. Moreover, the property for example of the histidine fusion portion, of itself being functionally active under denaturing conditions, is utilized. In the presence of a first solubilizing detergent, the denatured subunit is in step (a1) coupled for example to a nickel column, then the buffer is exchanged, and thus the first detergent dissolved therein is replaced by a buffer which comprises the folding-inducing detergent, after which the subunit bound to the column assumes its native structure. Then, in the subsequent step (b1), the natively folded subunit is eluted from the column for example by adding imidazole. It is optionally possible as stated above for a dialysis to follow, especially in order to remove free imidazole from the folded protein.

It is further preferred for the first detergent to be selected from the group comprising N-laurylsarcosine, urea, SDS (sodium dodecyl sulfate), guanidinium and/or FOS-Choline-14 (N-tetradecylphosphocholine).

This has the particular advantage that such highly active detergents ensure complete solubilization and denaturation of the expressed protein. Consequently, the precondition for substantially quantitative refolding of the ion channel protein into its native structure is set up thereby.

It is further preferred for the second detergent to be a milder detergent than the first, preferably N-tetradecylphosphocholine (FOS-Choline-14).

As the inventors have realized, this has the particular advantage that the desired refolding into the native structure of the ion channel protein takes place substantially quantitatively, and thus particularly efficiently, through the use of the milder detergent. In other words, replacement of the first detergent by a mild detergent, e.g. FOS-Choline-14, results in conversion of a large part of the originally functionally inactive ion channel protein into functionally active and natively folded ion channel protein.

In another embodiment, it is preferred for the same detergent to be employed as first and as second detergent, the second detergent being present in a lower concentration.

It is particularly preferred for the detergent to be N-tetradecylphosphocholine and to be employed as first detergent in a concentration of from 0.1 to 1%, preferably of 0.5%, and as second detergent in a concentration of from 0.01 to 0.5%, preferably of 0.05%.

In a further development of the method of the invention, the second detergent is present in a folding buffer in the form of mixed lipid/detergent micelles.

This has the particular advantage that stabilization of the ion channel proteins is better owing to these mixed micelles, i.e. after addition of lipids to the detergents. This in turn results in a further improvement in the yield of ion channel protein folded into its native structure through the use of lipid/detergent micelles.

It is further preferred for step (b) of the method of the invention to take place in a folding buffer which comprises a ligand of the ion channel or of a functional subunit thereof.

This has the particular advantage that this measure further increases the yield of naturely folded ion channel protein. In this case, the added ligand, for example glycine in the case of the glycine receptor channel, serves as “folding aid”, inducing a folding of the channel protein into the native structure and stabilizing the structure once folded.

It is preferred in one embodiment of the method according to the invention for the assembly of subunits of the ion channel into its active structure to take place in solution.

This can take place for example in the second detergent immediately after it has replaced the first detergent.

In another preferred embodiment, the assembly of subunits of the ion channel into its active structure takes place in proteoliposomes.

These comprise an aqueous compartment which is delimited by a bimolecular layer of lipids. This measure has the particular advantage that the incorporation of the folded protein in proteoliposomes results in a functional unit with which targeted investigation of certain processes on the channel proteins is possible. It is additionally possible for various channel subunits in proteoliposomes to be reconstituted or assembled to a complete ion channel, and for activity assays for example to be carried out with the latter.

Another object of the present invention therefore is a method for determining the activity of ligand-gated ion channels, comprising the steps of:
(a) provision of an ion channel which is present in its active structure in a suitable buffer,
(b) addition of a substance to be assayed, and
(c) determination of the ion flux,
   where the ion channel is prepared by the method according to the invention as described at the outset.

The substance can in this case be for example a ligand of the ion channel.

It is possible with said novel method to determine in this way for example the activity of an ion channel via the ability of the folded ion channel to bind a specific ligand. If the substance to be assayed binds to the ion channel, the latter opens, allowing an ion flux which can be determined by known techniques (for example patch clamp).

It is moreover possible with said novel method for example to screen for substances which block the ion channel.

A further advantage of the aforementioned novel method is that through the use of purified native and functionally active ion channels it is possible to employ larger and more homogeneous quantities of protein compared with ion channels present in membranes or with ion channels purified from membranes.

It is preferred in this connection for the ion channel to be present assembled in proteoliposomes.

It is possible to determine the ion flux easily in this way, so that the functioning ability of most of the ion channels of pharmacological interest can now also be tested.

In another embodiment of the method for determining the activity of ligand-gated ion channels or functional subunits thereof, it is preferred to carry out, instead of step (c), the step (c1) which determines the specific interaction of the ligand with the ion channel or a functional subunit thereof via measurement of the equilibrium constant K_{D}.

This constant describes the binding between ligand and ion channel. This has the particular advantage that this measure determines a quantity by which the activity of the ion channel or its binding ability is unambiguously quantified.

It is further preferred in another embodiment of the method for determining the activity of ligand-gated ion channels or functional subunits thereof to carry out instead of step (c) the step (c2) by which the specific interaction of the ligand with the ion channel or a functional subunit thereof is determined, via measurement of the competition of labeled ligand with unlabeled ligand for binding to the ion channel or a functional subunit thereof.

It is possible in this way to measure the activity and binding ability of the ion channel in a simple and accurate manner. In this procedure, a defined concentration of labeled ligand is added to the buffer. The complex of ion channel protein and labeled ligand can easily be detected by means of the label. Subsequently, unlabeled ligand is added in various concentrations to these mixtures. The labeled ligand is then displaced, depending on the concentration of unlabeled ligand, from the binding site on the ion channel. The remaining quantity of labeled ligands on the ion channel is subsequently measured. It is then easily possible from a large number of measurements to determine the equilibrium constant K_{D} on the basis of the known concentration of the two ligands.

Radiolabeled ligands are preferred in this connection, because in this way a radiolabel can easily be detected by means of established laboratory detection methods, e.g. autoradiography or scintillation counting.

Another object of the present invention is a method for determining the activity of ligand-gated ion channels or functional submits thereof, comprising the following steps:
(a) provision of ion channels present in proteoliposomes in their active structure, where the proteoliposomes are present in a suitable buffer and dyes are entrapped in the proteoliposomes,
(b) addition of a substance to be assayed, and
(c) determination of the color change,
   whereby the ion channel is prepared by the method according to the invention.

The substance can in this case be for example a ligand of the ion channel.

This method has the advantage that the activity of the ion channel can be determined visually. The ion channel to be assayed is reconstituted in the proteoliposomes. If a ligand which binds to the ion channel and thus opens it is then added, it is possible for example for substances to flow into the proteoliposomes, which are present in the buffer. The dyes which were protected from these substances in the proteoliposomes before the channels opened may thus cause for example a change in their fluorescence and cause a color change.

It is possible to employ as dye in this case for example anion-sensitive dyes such as, for example, 6-methoxy-N-(3-sulfopropyl)quinolinium (SPQ), N-(ethoxycarbonylmethyl)-6-methoxyquinolinium bromide (MQAE) or lucigenin. The three dyes differ in their excitation/emission wavelengths. It is possible with the method of the invention to determine in this way for example agonists and antagonists of the ligand.

As explained hereinbefore, the inventors have realized for the first time that said detergent induces a folding of functionally inactive or denatured and solubilized, for example in a strong detergent, ion channel protein into its functionally active or native structure. This realization makes it possible to use bacterial expression systems, which can be easily handled for preparing functionally active ion channel protein, because the inactive inclusion bodies can now be refolded by means of FOS-Choline-14 into active protein. This problem has not previously been solved in the art. In particular, the skilled person has lacked information on what type of and which detergent is suitable for inducing the refolding of denatured ion channel protein.

The inventors' achievement has also now made it possible to determine in a large-scale approach the activity of ion channels or of the subunits thereof, because this protein can now be provided in large quantities and in its native form.

Further advantages are evident from the exemplary embodiments hereinafter and the figures.

It will be appreciated that the features aforementioned and to be explained hereinafter can be used not only in the combinations indicated in each case but also in other combinations or alone without leaving the scope of the present invention.

### BRIEF DESCRIPTION OF THE FIGURE

The figure shows:

FIG. 1 the ability of the reconstituted α1-glycine receptor to bind strychnine;

### DESCRIPTION OF PREFERRED EMBODIMENTS

### EXAMPLE 1

### Bacterial Expression of Ion Channel Protein

The plasmids pBS-Glyα1 and pRC/CMV-Glyα1, which were obtained from the Neurobiology Institute, Heidelberg University, were the starting material.

The gene for the human (homomeric) glycine α1-channel was amplified by PCR under standard conditions and with use of primers with attached restriction cleavage site and Pfu polymerase (Stratagene, USA) from the plasmid pRC/CMV-Glyα1, and ligated via the restriction cleavage sites into the expression vector pGEX2aHis (modified pGEX2a vector from Pharmacia, Sweden). The ligation mixture was transformed into TOP10F′ cells (Invitrogen, Karlsruhe, Germany). Positive colonies were identified and sequenced using the standard sequencing primers pGEX5′ for/pBAD rev. It was possible to identify a positive clone by comparison with sequences from the EMBL Genbank (ACC number X52009). For expression, the plasmid pGEX2aHis-Glyα1 was transformed into BL21 cells (Novagen, USA).

200-400 ml of ampicillin-containing LB medium (10 g of Tryptone, 5 g of yeast extract, 10 g of NaCl) are inoculated with these transformed bacteria and incubated at 37° C. with shaking overnight. Subsequently, 20 ml of this preculture are introduced into one liter of ampicillin-containing LB medium and incubated at 37° C. with shaking until the optical density is OD=0.8.

Induction of expression of the fusion protein took place by addition of 100 μM IPTG (isoproyl β-thiogalactoside) to the culture. The culture is incubated for a further 3 hours. The culture is then centrifuged and the pellet from one liter is taken up in 100 ml of lysis buffer (10 mM Tris-HCl pH 7.5, 1 mM EDTA, 25% sucrose, 1 mM PMSF [phenylmethylsulfonyl], 1 mg/l lysozyme, 1 mM DTT).

### EXAMPLE 2

### Method with Laurylsarcosine/FOS-C-14

a) Solubilization of the Ion Channel Protein with Laurylsarcosine

The bacteria obtained from example 1 are disrupted using a microfluidizer under standard conditions. This results in a mixture of bacterial protein and of ion channel protein in the form of inclusion bodies.

In parallel, a nickel-NTA-Superflow column (Qiagen) is packed with 8 to 15 ml of column material and equilibrated with 5 column volumes of washing buffer (PBS, 1% laurylsacosyl [first detergent], 10 mM β-MSH) at a migration rate of about 2 to 10 ml/min.

The protein-containing solution is cautiously loaded in a volume of 200-500 ml at a constant migration rate of about 2 ml/min. The flow-through is passed through the column a second time.

After loading of the protein material, the column is washed with 8-15 column volumes of washing buffer. The migration rate can be increased for this to 10-20 ml/min. The bacterially expressed ion channel protein is now bound to the column material via its histidine fused portion. Bacterial protein is unable to bind to the column material and is removed by the washing step.

The bound ion channel protein is eluted from the column in a one-stage gradient. The elution buffer (PBS, 1% laurylsarcosine, 10 mM β-MSH, 300 mM imidazole) is loaded either continuously with a migration rate of 1-2 ml/min or discontinuously with a volume of 1 ml. Fractions of 1 ml are collected. The individual fractions are investigated for absorption at wavelengths from 320 nm to 240 nm in a photometer. Fractions with an absorption of >0.6 at 280 nm are collected and subsequently dialyzed together. The eluted ion channel protein is present in these fractions.

The pooled fractions are adjusted to a concentration of about 1 mg/ml protein. The total volume is determined and dialyzed against 100× the volume of washing buffer, but against at least 1 liter, overnight. This is followed by another dialysis against the same volume of washing buffer for 4 hours.

The volume of the dialyzed fraction is determined and the protein concentration is measured from the absorption at 280 nm.

This dialyzed fraction then contains the purified ion channel protein which has been solubilized and denatured in the first detergent (laurylsarcosine).

2b) Refolding of the Denatured Solubilized Ion Channel Protein Into Its Nature Structure

The refolding of the denatured ion channel protein is preferably carried out on a further Nickel-NTA-Superflow column (Qiagen). To do this, for example a mini column (MOBITEC, Göttingen, Germany) is packed with about 3-4 ml of column material and equilibrated with 5 column volumes of washing buffer (PBS pH 7.4, 1% laurylsarcosine, 10 mM β-MSH) at a migration rate of about 1-2 ml/min.

The column is closed at the outlet. The column is then filled up with the protein solution obtained from example 2 (about 10-12 ml, about 1 mg/ml protein). The column is subsequently closed completely and equilibrated in a rotator mixer in the cold room for 1 h.

The column material is then washed with twice the column volume of washing buffer. The migration rate through the column is then markedly reduced to slow to about 1 drop a second. The column is then washed with 5 column volumes of refolding buffer (25 mM potassium dihydrogen phosphate pH 7.4, 120 mM potassium gluconate, 1 mM GSH [glutathione], 0.2 mM GSSG [glutathione disulfide], 0.1% FOS-Choline-14 [second detergent], 0.01% deoxycholate, 0.01% shark lipid) with added ligand (in each case 200 mM for example of glycine, glutamate or GABA, depending on the receptor). The column outlet is then closed again and the column is filled up with refolding buffer. The column is subsequently incubated in a rotator at 4° C. for at least 2 h or else overnight. The ion channel protein which has then refolded into its native structure is now bound to the column material.

The refolding buffer is eluted from the column, and the column is washed again with 10 column volumes of refolding buffer—without ligands. Subsequently, refolding elution buffer (25 mM potassium dihydrogen phosphate pH 7.4, 120 mM potassium gluconate, 1 mM GSH, 0.2 mM GSSG, 0.1% FOS-Choline-14 [second detergent], 0.01% deoxycholate, 0.01% shark lipid, 0.5 M arginine, 0.5 M imidazole) with added ligand (in each case 200 mM for example of glycine, glutamate or GABA, depending on the receptor) is put on the column, and at least 12 fractions of 1 ml are collected. An absorption spectrum at 320 to 240 nm is recorded for the individual fractions. The fractions with an absorption of >0.5 at 280 nm are pooled and transferred into a 15 ml Falcon vessel. The absorption is again determined for the combined solution, and the protein concentration is adjusted with refolding elution buffer to 1 mg/ml.

The solution obtained in this way now contains the ion channel protein folded into its nature structure. Depending on the intended use of the protein, this can be followed by changing the buffer or dialysis.

### EXAMPLE 3

### Method with FOS-C-14/Different Concentrations

The solubilization buffer used in this case for the ion channel protein α1Gly-R prepared in example 1 and present in inclusion bodies (IB) was the following buffer: PBS (phosphate-buffered saline), pH 7.4; 0.5% FOS-C-14; 10 mM DTT (dithiothreitol). About 50 ml of buffer were employed in each case for 10 ml of IB. Before addition of the solubilization buffer, the IB were once again thoroughly homogenized in a Potter. The IB were then slowly added dropwise to the buffer. In order to solubilize as many of the IB as possible, the cloudy solution was optionally left to stir in a cold room at 4° C. for one to two hours and, once or twice during this, to sonicate with the ultrasonic tip at cycle 5/50% for 3 min.

This suspension was subsequently centrifuged in a Ti-45 (Beckmann) in an ultracentrifuge at 40 000 rpm at 4° C. for 20 min. The supernatant was diluted with 1:10 with PBS in order to reduce the DTT concentration to 1 mM and the FOS-C-14 concentration to 0.05%.

100 units of thrombin (Merck, Germany) were added per ml of IB, and the solution was incubated at room temperature for 4-5 h or optionally at 4° C. overnight. A further 30 units/ml of thrombin were to be added immediately before addition of Ni-NTA.

1 ml of Ni-NTA which had been equilibrated against PBS was added per ml of initial IB to the solution. The protein was incubated with Ni-NTA at 4° C. for at least two hours in the batch method, or the solution was pumped at a loading rate of 25 ml/h onto a previously prepared Ni-NTA column. The loading of the column was suspended until it was clear after the elution that the protein had specifically bound to the column and could be purified.

Nonspecifically bound proteins were washed out with a refolding washing buffer (PBS pH 7.4; 0.1% FOS-C-14; 20 mM imidazole; 0.01% deoxycholate; 0.01% shark lipid; 20 mM glycine; test pH again and titrate to 7.4) with at least 10-20 times the volume of the column.

Elution of His Proteins

The elution buffer used was optionally:

Elution buffer 1: PBS, pH 7.4; 0.1% FOS-C-14; 300 mM imidazole; 0.01% deoxycholate; 0.01% shark lipid; test pH again and adjust to 7.4.

Elution buffer 2: PBS, pH 7.4; 0.1% FOS-C-14; 300 mM imidazole; 0.01% deoxycholate; 0.01% shark lipid; 500 mM arginine hydrochloride; test pH again and adjust to 7.4.

Elution buffers 3 and 4 were made up with Tris/Cl/300 mM NaCl, pH 7.4, instead of PBS.

The individual fractions from the elution were measured at A₂₈₀ (spectrum from 320-240 nm). The samples with an absorption of >0.8 (undiluted) were collected and combined. The absorption of the combined fraction was then measured again and adjusted to a protein concentration of 1.3 mg/ml. The samples were diluted with the appropriate elution buffer for this purpose.

To stabilize the eluted protein fraction, optionally 100 mM glycine, 300 mM NaCl, 1 mM GSH/0.2 GSSG, 5 mM EDTA/5 mM EGTA, 20% sucrose, 15% glycerol, 50 mM sorbic acid, 100 μM strychnine was added. 10 mM MgCl₂ and 5 mM CaCl₂ were optionally added only in elution buffers 3 and 4. For screening, about 100 μl of protein solution were mixed with the appropriate stabilizers and incubated at 4° C. overnight.

### EXAMPLE 4

### Reconstitution of Native Ion Channels in Proteoliposomes

a) Liposome Preparation

Stock solutions of the three lipids cholesterol, 1-palmityl-2-oleylphosphatidylethanolamine (POPE) and 1-palmityl-2-oleylphosphatidylcholine (POPC) each of 100 mg/ml in chloroform are prepared. These are stored at −80° C. For use, the stock solutions are thawed in a exsiccator; the stock solutions must thereafter always be kept on ice.

A lipid mixture is prepared from the stock solutions by pipetting together 280 μl of cholesterol, 320 μl of POPE and 400 μl of POPC. This solution contains a total of 100 mg of lipids and is sufficient for reconstituting a maximum of 10 mg of protein.

The lipid mixture is put into a pear-shaped flask for the rotary evaporator, and 4 ml of chloroform are added per ml of lipid mixture. The mixture is introduced into the rotary evaporator. The mixture is mixed for 10 min. This is followed by incubation at 75 mbar for 30 min and a further incubation at 20 mbar, again for 30 min. The lipids, which are then dried, are taken up in 10 ml of double-distilled H₂O, corresponding to a final concentration of 10 mg/ml. The mixture is subsequently extruded through a 100 nm filter in an Avestin extruder. The liposomes can be stored at 4° C. for several weeks.

4b) Reconstitution of the Ion Channels

Complete ion channels are reconstituted by pipetting together a reconstitution mixture in a volume of 15 ml: 0.5-1.0 ml of protein solution from example 2b or example 3 (1 mg/ml), 1.5 ml of the previously prepared liposome solution (10 mg/ml) and 12 ml of reconstitution buffer (25 mM potassium dihydrogen phosphate pH 7.5, 120 mM potassium gluconate). The mixture is incubated at 20° C. in a rotator for about 24 h. During this time, the proteins or the individual ion channel subunits are taken up into liposomes in which they assemble to give a complete ion channel.

In order to remove any detergents present, which are derived from the protein preparation, examples 2b and 3, from the mixture, 2-3 ml of Calbiosorb material (Calbiochem, Darmstadt, Germany) are added to the reconstitution mixture and incubated at 20° C. for 4-24 h.

The Calbiosorb material is then removed on a 5 ml Mobitec column, and the flow-through is collected in ultracentrifugation centrifuge tubes (e.g. in tubes for the Ti-70 rotor, Beckmann).

The mixture is centrifuged in an ultracentrifuge at 55 000 rpm for 30 min. The supernatant is discarded and the pellet is thoroughly resuspended in 1 ml of reconstitution buffer; the mixture is then made up to 25 ml with reconstitution buffer.

This is followed by further ultracentrifugation at 60 000 rpm for 30 min. The supernatant is discarded and the pellet is thoroughly resuspended in 1 ml of assay buffer (for the glycine receptor channel: 25 mM potassium dihydrogen phosphate pH 7.5, 200 mM KCl; for the glutamate receptor channel 100 mM potassium dihydrogen phosphate pH 7.5, 0.5% BSA, 80 mM sucrose); the mixture is then made up to 25 ml with assay buffer.

This is followed by further ultracentrifugation at 60 000 rpm for 30 min.

The reconstituted ion channel is now present in proteoliposomes in the pellet. The pellet is taken up in 500 μl of assay buffer and thoroughly resuspended.

### EXAMPLE 5

### Determination of the Activity of a Reconstituted Ion Channel by Means of a Displacement Assay

Determination of the activity of an ion channel is described by way of example from the example of the reconstituted glycine receptor channel. The measure used for the activity in this case is the affinity of a ligand for its ion channel.

A radioactive displacement assay was carried out on the α1glycine receptor. The ligand employed for the glycine receptor channel is strychnine with a constant concentration of [³H]-strychnine and with a varying concentration of unlabeled strychnine. The property of the strychnine as a competitive antagonist to glycine is utilized in this case.

Stock solutions of 500 nM radioactive [³H]-strychnine and 5 mM “cold” strychnine, each in ethanol, are prepared. The activity assay is carried out on a 96-well MAFB NOB 50-plate (Millipore, Eschborn, Germany). The plate is coated with 200 μl of 0.3% polyethyleneimide and incubated at room temperature for 1 h. The plate is aspirated and washed twice with assay buffer (see example 4).

A first mixture consisting of 12 ml of assay buffer and 642 μl of [³H]-strychnine stock solution is prepared. This mixture is appropriately divided equally into 8 to 12 smaller mixtures. “Cold” strychnine is then pipetted, always in the same volume but in different concentrations, into these mixtures. The concentrations in this case should cover a range from 2 powers of ten below and 2 powers of ten above the binding constant or equilibrium constant K_{D} to be expected.

The ion channel was reconstituted in lipid vesicles as described in example 4. 20 μl of these proteoliposomes were employed for each data point. The binding mixtures were pipetted onto a 96-well glass fiber plate with a final volume of 150 μl and incubated at 20° C. for one hour. The concentration of [³H]-strychnine was set at a constant 20 nM. The concentration of “cold” strychnine was varied between 10⁻¹² and 10⁻⁷ molar in the respective binding mixture. The ligands bind to the ion channel protein; more of the respective strychnine is bound depending on the ratio of radiolabeled strychnine to “cold” strychnine.

In order to separate free ligand from protein-bound ligand, the binding mixture was filtered by suction through the 96-well glass fiber plate and washed twice with binding buffer (25 mM potassium phosphate, pH 7.4; 200 mM potassium chloride) at 4° C. The proteoliposomes and the ligand bound thereto was retained by the glass fiber membrane. The membranes in the 96-well plate were dried, and the individual wells were filled up 50 μl of scintillation cocktail. The radioactivity (CPM) remaining on the filter was converted into picomolar of bound [³H]-strychnine.

For control measurements, lipid vesicles were incubated with radiolabeled and “cold” ligands and treated in the same way.

The concentration of “cold” strychnine which leads to half the maximum decrease in the binding ability of radioactive strychnine was subsequently determined.

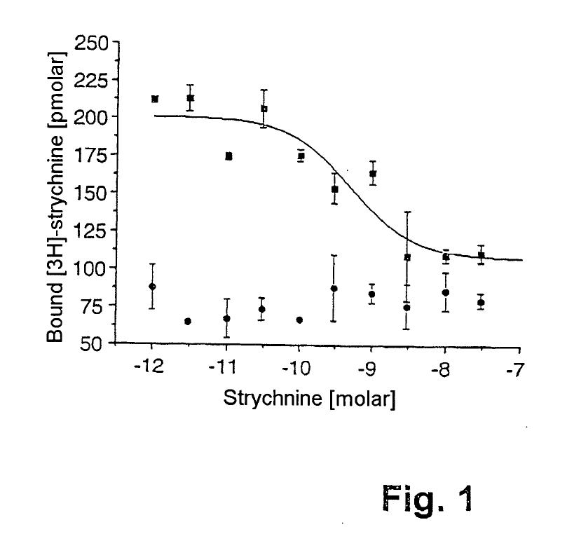

The displacement curve for [³H]-strychnine/unlabeled strychnine on α1Gly receptor proteoliposomes is depicted in FIG. 1. The concentration of bound [³H]-strychnine is plotted as a function of the respective concentration of cold strychnine for α1Gly receptor proteoliposomes (black squares) and empty lipid vesicles (black circles). Each data point in FIG. 1 was averaged from three independent measurements. The displacement curve was generated with the aid of the Origin 6.0 software version as one-site competition model. The pI₅₀ value represents the turning point in the curve profile of the displacement curve and corresponds in each case to the Kd.

### EXAMPLE 6

### Determination of the Activity of a Reconstituted Ion Channel by Measuring Ion Currents

The ion channels reconstituted in liposomes as in example 4 were subjected to a measurement of the ion current. For this purpose, the proteoliposomes were mixed with the following extracellular solution: buffer 2 pipette solution: 140 mM KCl, 10 mM EGTA, 10 mM HEPES, 28 μM glycine.

The ion currents were measured using the patch clamp technique, carrying out test mixtures without glycine as controls.

### EXAMPLE 7

### Preparation of Proteoliposomes Comprising Reconstituted Ion Channels with Dyes Entrapped Therein

Dyes which can be employed are for example anion-sensitive dyes such as, for example, 6-methoxy-N-(3-sulfopropyl)quinolinium (SPQ), N-(ethoxycarbonylmethyl)-6-methoxyquinolinium bromide (MQAE) or lucigenin. The three dyes differ in their excitation/emission wavelengths.

The proteosomes are prepared as described in example 4.

It is possible to employ as lipid mixture in this case in particular also a mixture of 40% soybean phosphatidylcholine (PC), 32% sheep's brain phosphatidylethanolamine (PE) and 28% cholesterol. The lipid is taken up in a concentration of 100 mg/ml in 120 mM potassium gluconate, 25 mM potassium hydrogen phosphate, pH 7.4. The lipid is where appropriate extruded 19 times through 200 nm filters.

Entrapment of the anion-sensitive dyes can take place in various ways, for example spontaneously, by alternating freezing and thawing, by extrusion or by sonication.

The fluorescence of SPQ, MQAE and lucigenin is strongly quenched by iodide. Dye-loaded proteoliposomes are added to quenching buffer (40 mM potassium iodide, 80 mM potassium gluconate, 25 mM potassium hydrogenphosphate, pH 7.4). Excitation in a fluorometer leads to a fluorescence signal. Addition of a ligand (for example strychnine) opens the ion channels and iodide can flow into the proteoliposomes. This leads to a decrease in the fluorescence.

1. A method for preparing ion channels which are folded into their active structure, or functional subunits thereof, comprising the steps of:
(a) provision of expressed subunits of an ion channel which are solubilized and denatured in a first detergent,
(b) replacement of the first detergent by a second detergent which induces folding of the subunits of an ion channel into their native structure, and
(c) assembly of subunits of the ion channel into its active structure.

2. The method as claimed in claim 1, wherein the ion channel is a voltage-gated ion channel.

3. The method as claimed in claim 1, wherein the ion channel is a ligand-gated ion channel.

4. The method as claimed in claim 1, wherein the subunits are homomers.

5. The method as claimed in claim 1, wherein the subunits are heteromers.

6. The method as claimed in claim 1, wherein in step (a) the provision takes place via expression of the subunits as fusion proteins.

7. The method as claimed in claim 1, wherein step (a) is followed by a step (a1) in which the subunit is bound to a chromatography column material and step (b) is followed by a step (b1) in which the subunit is eluted from the chromatography column material.

8. The method as claimed in claim 1, wherein the first detergent is selected from the group consisting of: N-laurylsarcosine, urea, sodium dodecyl sulfate (SDS), guanidinium and/or N-tetradecylphosphocholine (FOS-Choline-14).

9. The method as claimed in claim 1, wherein the second detergent is a mild detergent.

10. The method as claimed in claim 1, wherein the second detergent is N-tetradecylphospho-choline (FOS-Choline-14).

11. The method as claimed in claim 1, wherein the same detergent is employed in each case as first and as second detergent, the second detergent being present in a lower concentration than the first.

12. The method as claimed in claim 11, wherein the detergent is N-tetradecylphosphocholine and is employed as first detergent in a concentration of from 0.1 to 1%, and as second detergent in a concentration of from 0.01 to 0.5%.

13. The method as claimed in claim 11, wherein the detergent is N-tetradecylphosphocholine and is employed as first detergent in a concentration of 0.5%, and as second detergent in a concentration of 0.05%.

14. The method as claimed in claim 1, wherein the second detergent is present in a folding buffer in the form of mixed lipid/detergent micelles.

15. The method as claimed in claim 1, wherein step (b) takes place in a folding buffer which comprises a ligand of the ion channel or of a functional subunit thereof.

16. The method as claimed in claim 1, wherein the assembly of subunits of the ion channel into its active structure takes place in solution.

17. The method as claimed in claim 1, wherein the assembly of subunits of the ion channel into its active structure takes place in proteoliposomes.

18. A method for determining the activity of ligand-gated ion channels or functional subunits thereof, comprising the steps of:
(a) provision of an ion channel which is present in its active structure in a suitable buffer,
(b) addition of a ligand of the ion channel, and
(c) determination of the ion flux,
wherein the ion channel is prepared by the method as claimed in claim 3.

19. The method as claimed in claim 18, wherein the ion channel is present in proteoliposomes.

20. The method as claimed in claim 18, wherein instead of step (c) the step (c1) is carried out by which the specific interaction of the ligand with the ion channel or a functional subunit thereof is determined via measurement of the equilibrium constant K_{D}.

21. The method as claimed in claim 16, wherein instead of step (c) the step (c2) is carried out by which the specific interaction of the ligand with the ion channel or a functional subunit thereof is determined via measurement of the competition of labeled ligand with unlabeled ligand for binding to the ion channel or a functional subunit thereof.

22. A method for determining the activity of ligand-gated ion channels or functional subunits thereof, comprising the steps of:
(a) provision of ion channels present in proteoliposomes in their active structure, where the proteoliposomes are present in a suitable buffer and dyes are entrapped in the proteoliposomes,
(b) addition of a ligand of the ion channel, and
(c) determination of the color change,
wherein the ion channel is prepared by the method as claimed in claim 3.
